(19) United States
(12) Patent Application Publication
(10) Pub. No.: US 2026/0207140 A1
(43) Pub. Date: Jul. 23, 2026

(54) FLEXIBLE BIOSENSOR WITH MICRONEEDLE ARRAY AND APPLICATION THEREOF

(71) Applicant: Beijing Information Science and Technology University, Beijing (CN)

(72) Inventors: Yanxia Chen, Beijing (CN); Xingping Zhong, Beijing (CN); Lei Qin, Beijing (CN)

(21) Appl. No.: 19/263,452

(22) Filed: Jul. 8, 2025

(30) Foreign Application Priority Data
Jan. 22, 2025 (CN) 202510104666.X

(51) Int. Cl. A61B 5/00; A61B 5/145

(52) U.S. Cl. CPC A61B 5/685; A61B 5/14517; A61B 5/4266; A61B 5/6832; A61B 2562/046; A61B 2562/12; A61B 2562/164

(57) ABSTRACT

Disclosed is a flexible biosensor with a microneedle array, falling within the technical fields of flexible wearable sensing, biosensing, and medical detection. The biosensor includes a hydrophilic microneedle array and a hydrophobic flexible substrate. The flexible substrate includes hydrophilic detection zones based on the microneedle array and a hydrophobic background surface of the flexible substrate. Both the flexible substrate and the hydrophilic microneedle array are fabricated from flexible materials, with the microneedle array exhibiting hydrophilicity to facilitate the directional transportation of sweat. The hydrophilic detection zones constructed based on these microneedles, combined with the hydrophobic background surface of the flexible substrate, are capable of facilitating sweat enrichment in a sensor detection area. Functional modification of this detection area is capable of allowing for specific site-directed detection of sweat.

### CROSS-REFERENCE TO RELATED APPLICATION

This application claims priority of Chinese Patent Application No. 202510104666.X, filed on Jan. 22, 2025, the entire contents of which are incorporated herein by reference.

### TECHNICAL FIELD

The present disclosure belongs to the technical fields of flexible wearable sensing, biosensing, and medical detection, and in particular to a flexible biosensor with a microneedle array and an application thereof.

### BACKGROUND

Sweat, a fluid secreted by sweat glands, is primarily composed of water and contains rich physiological information from human body, such as various electrolyte ions (e.g., potassium, calcium, sodium, and chloride) and metabolites produced by the body (e.g., glucose, lactate, uric acid, urea, etc.). Numerous clinical medical studies indicate that dysregulation of the human body's physiological functions can lead to changes in the composition of sweat. For instance, elevated blood glucose levels can cause an increase in sweat glucose concentration; cystic fibrosis can result in sweat with elevated salt content; and drug abuse can lead to the presence of corresponding metabolites in sweat. Furthermore, sweat does not require invasive extraction from the human body. Therefore, sweat is a valuable and non-invasive subject for detection purposes.

At present, sweat detection still faces several challenges. In terms of substrate materials for sweat sensing interfaces, sweat sensors made from purely rigid substrates are not easily stretchable and lack comfort and wearability. On the other hand, most sweat detection involves collecting sweat from the skin surface and sending it to a laboratory for testing, which prevents rapid real-time detection and timely feedback. Additionally, sweat itself is characterized by properties that hinder detection, such as its small volume, dispersed nature, rapid evaporation rate, susceptibility to contamination from skin surface impurities, and low concentration of components. These characteristics pose significant challenges to sweat collection.

Therefore, it is essential to design a flexible wearable sweat sensor that integrates sweat transport, enrichment, and real-time detection.

### SUMMARY

One objective of the present disclosure is to provide a flexible biosensor with a microneedle array. The biosensor is fabricated from flexible materials, and includes a hydrophilic microneedle array and a hydrophobic flexible substrate; and the flexible substrate includes hydrophilic detection zones based on the microneedle array and a hydrophobic background surface of the flexible substrate. Both the flexible substrate and the hydrophilic microneedle array are fabricated from flexible materials, with the microneedle array exhibiting hydrophilicity to facilitate the directional transportation of sweat. The hydrophilic detection zones constructed based on these microneedles, combined with the hydrophobic background surface of the flexible substrate, can facilitate sweat enrichment in a sensor detection area. Functional modification of this detection area can enable specific site-directed detection of sweat.

Preferably, the flexible materials are selected from one or more of the following: polydimethylsiloxane (PDMS), silica gel, hydrogel, silicone rubber, and epoxy resin. These materials can also be doped with magnetic or responsive materials to achieve specialized functionalization.

Preferably, a preparation method for the biosensor includes one or more techniques selected from micromolding, three-dimensional (3D) printing, etching, replication, hot embossing, and self-assembly. More preferably, surfaces of the hydrophilic microneedle array and the hydrophilic detection zones are either smooth, or fabricated with microstructural textures/microgrooves, or possess micro/nano-scale structured features.

More preferably, the structures constructed on the surfaces of the hydrophilic microneedle array and the hydrophilic detection zones are fabricated through deposition, etching, evaporation, spray coating, sandblasting, spin coating, blade coating, or mineralization.

More preferably, structural materials for the surfaces of the hydrophilic microneedle array and the hydrophilic detection zones are selected from one or more of the following: metal-organic frameworks (MOFs), metal oxides, non-metallic oxides, or mineral-based micro/nanomaterials.

Preferably, the hydrophilic microneedle array is either vertically oriented or arranged at a specific inclined angle.

Another objective of the present disclosure is to provide a preparation method for the flexible biosensor with a microneedle array, including the steps of:
S1, fabricating a flexible substrate and a microneedle array using one or more techniques selected from micromolding, 3D printing, etching, replication, hot embossing, and self-assembly;
S2, further constructing microstructural textures/microgrooves or micro/nano-scale structured features on surfaces of the microneedle array and detection zones;
S3, performing functional modification on the surfaces of the detection zones of the microneedle array; and
S4, incorporating a skin adhesion layer.

Preferably, the flexible substrate exhibits hydrophobic properties; and the hydrophilic microneedle array can be designed as required, for example, as a 4*4 array or a 2*3 array; dimensions of microneedle length, taper angle, base diameter, and spacing can be customized according to specific requirements, such as microneedle length: 0.5 mm, 1.5 mm, or 2.5 mm (height), taper angle: 20°, 30°, or 45°, base diameter: 1.3 mm or 1.0 mm, and spacing: 3.0 mm or 5.0 mm; inclination angles of microneedles can also be customized according to specific requirements, such as 60°, 45°, or 30°; and detection zones with micro/nano-scale structured features can be designed and prepared as required, including the steps of sequentially adding 0.15 g of zinc nitrate hexahydrate, 0.07 g of triethylamine hydrochloride, and 0.12 g of 2-methylimidazole to 50 mL of deionized water for deposition to form micro/nanostructures.

A further objective of the present disclosure is to provide an application of the biosensor in preparation of products for sweat or glucose detection.

Compared to the relate art, the present disclosure has the following beneficial effects.

In the present disclosure, the microneedle array-based biosensor is constructed using the flexible materials, incorporating hydrophobic/hydrophilic modification and functional modification. The hydrophilic microneedles can facilitate sweat transport, while the detection zones constructed based on these hydrophilic microneedles, combined with the hydrophobic background surface of the flexible substrate, can facilitate sweat enrichment in the sensor detection area. The detection area that has undergone functional modification can allow specific detection of various physiological indicators in sweat. Through the synergistic effects of microneedle-mediated transport and enrichment, sensitive detection of trace samples is achieved.

In a further description, surfaces of the microneedle array in the present disclosure may incorporate micro/nanostructures that facilitate sweat transport, enrichment, and sensitive detection. The present disclosure integrates sweat transport, enrichment, and detection, featuring low fabrication costs, wearable comfort, and non-invasive, painless operation. It can perform simultaneous detection of one or multiple physiological indicators, making it suitable for point-of-care testing of human sweat.

### BRIEF DESCRIPTION OF THE DRAWINGS

FIG. 1 is a schematic structural diagram of a flexible biosensor fabricated in Embodiment 1.

FIG. 2 is a characterization diagram of a microneedle on the flexible biosensor in Embodiment 1.

FIG. 3 is a contact angle image showing hydrophobicity of a flexible background substrate in Embodiment 3.

FIG. 4 is a contact angle image showing hydrophilicity of a microneedle array with micro/nanostructures and a detection zone in Embodiment 3.

FIG. 5 is a diagram showing a transportation process of sweat in Embodiment 3.

FIG. 6 is a curve graph showing pH detection on the microneedle array-based flexible biosensor in Embodiment 4.

FIG. 7 is a curve graph showing glucose detection on the microneedle array-based flexible biosensor in Embodiment 6.

FIG. 8 is a linear fitting curve graph of a green (G) value in the glucose detection in Embodiment 7.

### DETAILED DESCRIPTION

### Embodiment 1: Fabrication of a Flexible Substrate With a Vertical Microneedle Array

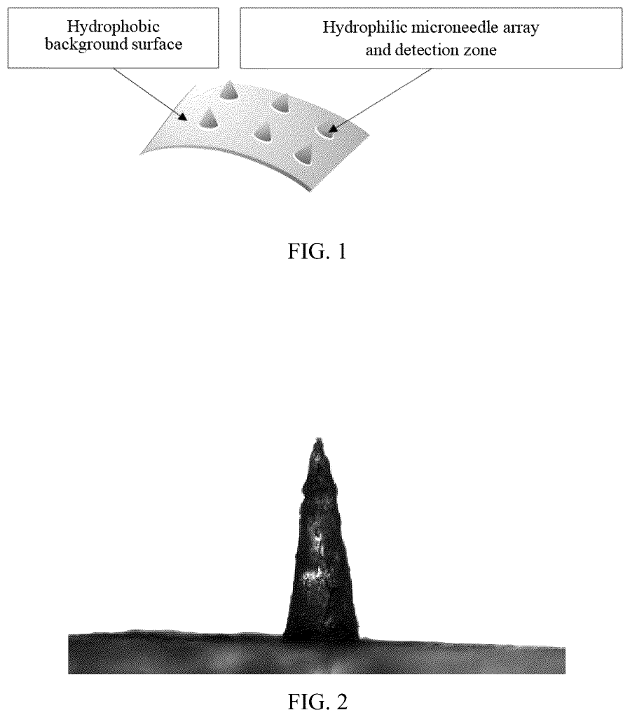

1. A flexible substrate and a microneedle array were formed by PMDS. After thoroughly mixing a prepolymer (Sylgard 184) and a curing agent (Sylgard 184) at a mass ratio of 10:1, the mixture was poured into a vertical microneedle array mold (needle height: 2.0 mm, base diameter: 0.9 mm, 2*3 array configuration, needle spacing: 5.0 mm, needle taper angle: 25°). The mold was placed in a vacuum tank and evacuated to −0.08 MPa. This evacuation process was repeated 3-5 times. Following a standing period for 1 h, the mold was transferred to an electric thermostatic drying oven and cured at 65° C. for 2 h. The cured PDMS was demolded to obtain the flexible substrate with a vertical microneedle array.
2. 0.15 g of zinc nitrate hexahydrate, 0.07 g of triethylamine hydrochloride, and 0.12 g of 2-methylimidazole were sequentially added to 50 mL of deionized water, and each component was stirred for 10 min using a magnetic stirrer to ensure thorough mixing.
3. The prepared flexible substrate with a vertical microneedle array was sequentially placed in deionized water, 75% of alcohol and ultrapure water, undergoing ultrasonic cleaning for 10 min in each solution, and a photomask (thickness: 0.1 mm) featuring specific patterns was covered on the prepared flexible substrate with a vertical microneedle array.
4. The solution prepared in step 2 was first added into a clean weighing bottle, a sensor substrate covered with the photomask was placed into the solution, and the weighing bottle was placed into the electric thermostatic drying oven at 80° C., undergoing deposition for 24 h.
5. The sensor substrate covered with the photomask was removed, and the photomask was carefully detached from the flexible substrate and the microneedle array. The substrate was allowed to air-dry at room temperature to obtain a flexible substrate with a vertical microneedle array featuring micro/nanostructures. A schematic diagram is shown in FIG. 1, and a vertical microneedle is depicted in FIG. 2.

### Embodiment 2: Fabrication of a Flexible Substrate With an Inclined Microneedle Array

1. A flexible substrate and a microneedle array were formed by PMDS. After thoroughly mixing a prepolymer and a curing agent at a mass ratio of 10:1, the mixture was poured into an inclined microneedle array mold (needle height: 3.0 mm, base diameter: 1.0 mm, inclination angle: 60°, 6*6 array configuration, needle spacing: 8.0 mm, taper angle: 30°). The mold was placed in a vacuum tank and evacuated to −0.09 MPa. This evacuation process was repeated 3-5 times. Following a standing period for 30 min, the mold was transferred to an electric thermostatic drying oven and cured at 100° C. for 1 h. The cured PDMS was demolded to obtain the flexible substrate with an inclined microneedle array.
2. 0.15 g of zinc nitrate hexahydrate, 0.07 g of triethylamine hydrochloride, and 0.12 g of 2-methylimidazole were sequentially added to 50 mL of deionized water, and stirred for 30 min using a magnetic stirrer to ensure thorough mixing.
3. The prepared flexible substrate with an inclined microneedle array was sequentially placed in deionized water, 75% of alcohol and ultrapure water, undergoing ultrasonic cleaning for 10 min in each solution, and a photomask featuring specific patterns was covered on the prepared flexible substrate with an inclined microneedle array, and cleaned in a plasma cleaner for 5 min.
4. The solution prepared in step 2 was first added into a clean weighing bottle, a sensor substrate covered with the photomask in step 3 was placed into the solution, and the weighing bottle was placed into the electric thermostatic drying oven for deposition for 8 h.
5. The sensor substrate covered with the photomask was removed. Following a standing period to air-dry at room temperature, the photomask carefully detached from the flexible substrate and the inclined microneedle array, yielding a flexible substrate with an inclined microneedle array.

### Embodiment 3: Sweat Transport Process of a Flexible Substrate With a Microneedle Array

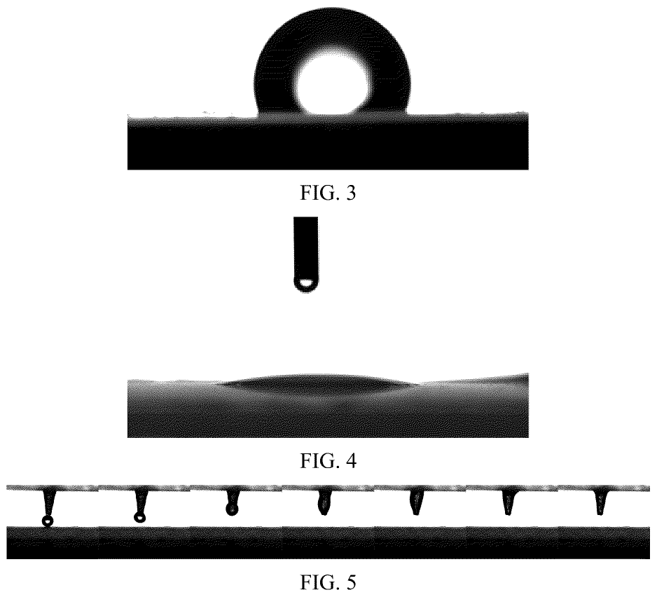

The flexible PDMS background substrate prepared in Embodiment 1 exhibited hydrophobicity, as shown in FIG. 3, with a water contact angle of approximately 106.7°±2.8° on this flexible background surface. In contrast, the obtained microneedle array and detection zones had the micro/nanostructure and demonstrated hydrophilicity, as shown in FIG. 4, exhibiting a water contact angle of about 15.6°±4.0°. Using this sensor substrate prepared in Embodiment 1, when the microneedle was brought into contact with 1 μL of sweat, the sweat was adsorbed onto the microneedle tip and transport along the microneedle, as shown in FIG. 5.

### Embodiment 4: pH Detection

Functional modification of the microneedle array detection zones: using the sensor substrate prepared in Embodiment 1, 1 μL of purple litmus solution was dispensed onto each microneedle array detection zone. Upon solvent evaporation, components used for pH detection were loaded onto the microneedle array detection zones, thereby completing the functional modification.

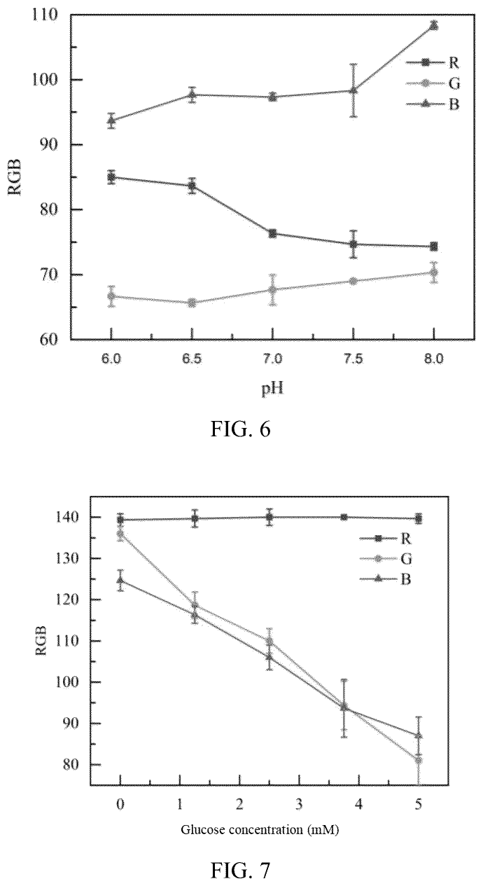

PH detection: the above functionalized microneedle array was employed to detect 1 μL of pH standard solutions (with pH values of 6.0, 6.5, 7.0, 7.5, and 8.0, respectively). The pH sensor was placed in a water bath maintained at 37° C. for 3 min, followed by removal after completion of the reaction. Under controlled lighting conditions, red-green-blue (RGB) values of a colorimetric response were captured, extracted, and recorded using a smartphone, and variation curves under different pH conditions were plotted, as shown in FIG. 6.

### Embodiment 5: Human Sweat pH Detection

Functional modification of the microneedle array detection zones: using the sensor substrate prepared in Embodiment 1, 1 μL of purple litmus solution was dispensed onto each microneedle array detection zone, and upon solvent evaporation, the component used for pH detection was loaded onto the microneedle array detection zone, thereby completing the functional modification.

Integration with skin adhesion layer: the fabricated flexible sensor substrate with a microneedle array was fixed onto an adhesive medical bandage to ensure wearable detection. The flexible sensor was affixed to a forearm of a test subject, causing the subject to engage in physical exercise. After perspiration occurred, the RGB values from the microneedle array detection zones were captured, extracted, and recorded using the smartphone. These values were compared with the concentration fitting curve (as shown in FIG. 6) to derive the corresponding human sweat pH value.

### Embodiment 6: Glucose Concentration Detection

Functional modification of the microneedle array detection zones: using the sensor substrate prepared in Embodiment 1, 1 μL of working solution (prepared by mixing phenol reagent and enzyme reagent in a 1:1 volume ratio) from a glucose assay kit (KB 016-200T, BIOISCO, China) was dispensed onto each microneedle array detection zone. Upon solvent evaporation, active components used for glucose concentration detection were loaded onto the microneedle array detection zones, thereby completing the functional modification.

Glucose detection: clean test tubes were prepared, and predetermined amounts of anhydrous glucose and deionized water were added to each tube to prepare glucose standard solutions with concentrations of 0 mM, 1.25 mM, 2.50 mM, 3.75 mM, and 5.00 mM. These solutions were set aside for subsequent use. The above functionalized microneedle array was employed to detect 1 μL of glucose standard solutions. The sensor was placed in a water bath maintained at 37° C. for 4 min, followed by removal after completion of the reaction. Under controlled lighting conditions, RGB values of a colorimetric response were captured, extracted, and recorded using a smartphone, and variation curves under different glucose concentrations were plotted, as shown in FIG. 7.

### Embodiment 7: Accuracy Validation of Glucose Detection

Functional modification of the microneedle array detection zones: using the sensor substrate prepared in Embodiment 1, 1 μL of working solution from the glucose assay kit was dispensed onto each microneedle array detection zone. Upon solvent evaporation, the active components used for glucose concentration detection were loaded onto the microneedle array detection zones, thereby completing the functional modification of sensor.

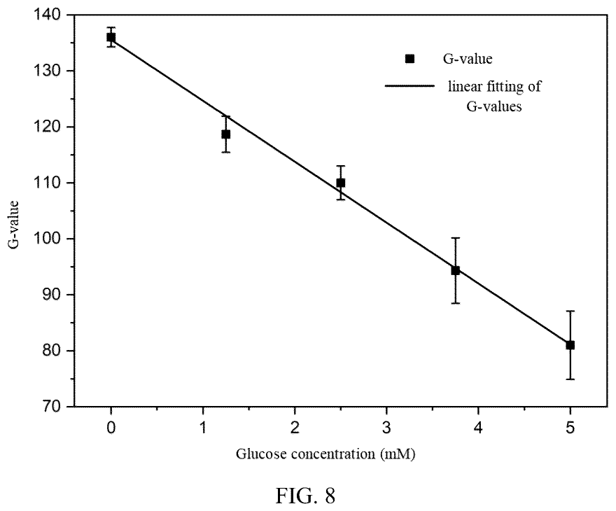

Validation: a linear fitting curve of the relationship between the color changes (captured in the green channel with obvious color change, G-value) and the concentration was plotted as shown in FIG. 8. A linear fitting equation was y=−10.88x+135.55, with R²=0.987. The clean test tube was prepared, and predetermined amounts of anhydrous glucose and deionized water were added to the tube to prepare a glucose standard solution with concentration of 1.00 mM. The solution was set aside for subsequent use. The above sensor with functionalized microneedle array was employed to detect 1 μL of the glucose standard solution. The sensor was placed in the water bath maintained at 37° C. for 4 min, followed by removal after completion of the reaction. Under controlled lighting conditions, the smartphone was used for capturing an image, extracting, and recording the G-value of the colorimetric reaction as 124. This value was substituted into the equation to obtain a glucose concentration of 1.062 mM, with an error of approximately 6.2%.

### Embodiment 8: The Detection of Sweat Glucose

Functional modification of the microneedle array detection zones: using the sensor substrate prepared in Embodiment 1, 1 μL of working solution from the glucose assay kit was dispensed onto each microneedle array detection zone. Upon solvent evaporation, the active components used for glucose concentration detection were loaded onto the microneedle array detection zones, thereby completing the functional modification.

Integration with skin adhesion layer: the fabricated flexible sensor substrate with a microneedle array was fixed onto an adhesive medical bandage to ensure wearable detection. The flexible sensor was affixed to a forearm of a test subject and allowed the subject to have a meal. After the meal, the RGB values from the microneedle array detection zones were captured, extracted, and recorded using the smartphone. The G-values were compared with the concentration fitting curve (as shown in FIG. 8) to derive the corresponding glucose concentration.

The foregoing embodiments are merely to describe preferred implementations of the present disclosure, rather than limiting the scope of the present disclosure. Without departing from the design spirit of the present disclosure, any modifications or improvements to the technical solutions of the present disclosure made by a person skilled in the art shall fall within the scope of protection defined by the claims of the present disclosure.

1. A flexible biosensor with a microneedle array, fabricated from flexible materials, and comprising a hydrophilic microneedle array and a hydrophobic flexible substrate, the flexible substrate comprising hydrophilic detection zones based on the microneedle array and a hydrophobic background surface of the flexible substrate.

2. The flexible biosensor according to claim 1, wherein the flexible materials are selected from one or more of the following: polydimethylsiloxane (PDMS), silica gel, hydrogel, silicone rubber, and epoxy resin.

3. The flexible biosensor according to claim 1, wherein a preparation method for the biosensor comprises one or more techniques selected from micromolding, three-dimensional (3D) printing, etching, replication, hot embossing, and self-assembly.

4. The flexible biosensor according to claim 1, wherein surfaces of the hydrophilic microneedle array and the hydrophilic detection zones are either smooth, or fabricated with microstructural textures/microgrooves, or possess micro/nano-scale structured features.

5. The flexible biosensor according to claim 4, wherein the structures constructed on the surfaces of the hydrophilic microneedle array and the hydrophilic detection zones are fabricated through deposition, etching, evaporation, spray coating, sandblasting, spin coating, blade coating, or mineralization.

6. The flexible biosensor according to claim 5, wherein the structures constructed on the surfaces of the hydrophilic microneedle array and the hydrophilic detection zones are formed by one or more materials selected from metal-organic frameworks (MOFs), metal oxides, non-metallic oxides, or mineral-based micro/nanomaterials.

7. The flexible biosensor according to claim 1, wherein the hydrophilic microneedle array is either vertically oriented or arranged at a specific inclined angle.

8. A preparation method for the flexible biosensor with a microneedle array, comprising the steps of:
S1, fabricating a microneedle array and a flexible substrate using one or more techniques selected from micromolding, 3D printing, etching, replication, hot embossing, and self-assembly;
S2, further constructing microstructural textures/microgrooves or micro/nano-scale structured features on surfaces of the microneedle array and detection zones;
S3, performing functional modification on the surfaces of the detection zones of the microneedle array; and
S4, incorporating a skin adhesion layer.

9. The preparation method according to claim 8, wherein the flexible substrate exhibits hydrophobic properties; and microneedles are arranged in an X*Y array.

10. An application of a flexible biosensor in preparation of wearable products for sweat or glucose detection according to claim 1.

11. An application of a flexible biosensor in preparation of wearable products for sweat or glucose detection according to claim 2.

12. An application of a flexible biosensor in preparation of wearable products for sweat or glucose detection according to claim 3.

13. An application of a flexible biosensor in preparation of wearable products for sweat or glucose detection according to claim 4.

14. An application of a flexible biosensor in preparation of wearable products for sweat or glucose detection according to claim 5.

15. An application of a flexible biosensor in preparation of wearable products for sweat or glucose detection according to claim 6.

16. An application of a flexible biosensor in preparation of wearable products for sweat or glucose detection according to claim 7.
